(19) United States
(12) Patent Application Publication
(10) Pub. No.: US 2006/0165643 A1
(43) Pub. Date: Jul. 27, 2006

(54) Cosmetic or dermopharmaceutical compositions containing kombucha

(76) Inventor: Karl Lintner, Rambouillet (FR)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 10/523,333

(22) PCT Filed: Jul. 25, 2003
(86) PCT No.: PCT/FR03/02368

§ 371 Date: Aug. 29, 2005

(30) Foreign Application Priority Data
Jul. 30, 2002 (FR) 02/09710

(51) Int. Cl. A61K 36/82; A61K 9/127; A61K 8/97

(52) U.S. Cl. 424/74; 424/729; 424/450; 977/907

(57) ABSTRACT

Cosmetic or dermopharmaceutical compositions which contain kombucha are disclosed. The present invention further relates to the use of kombucha and cosmetic or dermopharmaceutical compositions containing the same, alone or in combination, for the care of the skin, mucosae and skin appendages, and in particular for preventing the signs of endogenous and/or exogenous ageing.

The present invention relates to cosmetic or dermopharmaceutical compositions containing kombucha and the use of kombucha and cosmetic or dermopharmaceutical compositions containing the same, alone or in combination, for the care of the skin, mucosae and skin appendages, and in particular for preventing the signs of endogenous and/or exogenous ageing. The skin, mucous membranes, and the skin appendages are fragile elements which deserve the greatest regards. Indeed, their balance is disturbed permanently by external aggressions, sun, wind, cold. The skin ageing is one of the first external signs of ageing. It results from a certain number of deteriorations which occur spontaneously in the course of the time but which can also be induced by external factors (solar radiations, tobacco smoke, excessive alcohol consumption).

The signs of ageing result clinically by the appearance of wrinkles and small lines, in a slackening of the cutaneous and subcutaneous tissues which result in a dull texture of skin, a slackening of the cutaneous microrelief, a decreased cutaneous firmness, a loss of elasticity, and an overall flask, duller skin and without freshness of the complexion.

Ageing also results in a reduction in the number, thickness and growth of the hair, a fall of secretions (sebaceous and sweat), and a deterioration of the structure and growth of the nails. On the parts of the skin which were exposed to the sun throughout the life—primarily the face, the low neckline, hands and before arm—one often observes spots of hyper- or hypo-pigmentation.

Some of these signs are more particularly related to intrinsic or physiological ageing, i.e. with ageing related to the age, whereas others are more specific of extrinsic ageing, i.e. ageing caused generally by the environment; it is more particularly about photo-ageing due to the exposure to the sun, the light or any other radiation.

The present invention is interested in the general treatment of the ageing of the skin, the mucous membranes and the skin appendages, in particular by the improvement of the freshness of the complexion, and in particular by the prevention or the deceleration of the glycation, as well as by the stimulation of the lipids synthesis by the cutaneous adipocytes.

There are two types of collagen glycosilation. One, enzymatic, natural and necessary, is a step of the formation of collagen. The other, which is an aspecific glycation, is a chemical reaction which occurs spontaneously (without the intervention of an enzymatic system), a little everywhere in the organism, between glucose and many biochemical components such as long lifespan proteins [THORPE S R, 1996; NAGARAJ R H, 1996]. These spontaneous reactions are known under the name of reaction of Maillard. The principal physiological causes currently known as responsible for this phenomenon are diabetes (because of the abnormally high rate of blood glucose) and ageing. Pathologies which rise from the glycation in the case of the diabetes are in particular the cataract, various neuropathies, renal attacks, micro- and macro-angiographies. In the case of ageing, the complications most often met reach collagen: with the age, the mechanical resistance (with the stretching) of collagen increases. The glycosylation of collagen fibres and elements of conjunctive tissue in general, by creating definitive reticulations between various fibres, involving their rigidification, and from there a loss of suppleness and elasticity of the skin.

The age advancing, the skin cells (and in particular collagen fibres which are the principal one constituting extracellular matrix) are renewed less better and less quickly. The derm is getting thinner, but the surface layer thickens by a defective elimination of the dead cells.

Within these observations, one deduces from it that the glycation deceleration of cutaneous collagen will make it possible to the skin to preserve a suppleness and a much better elasticity, thus improving his aspect and the feeling of comfort.

The invention being the subject of this patent application thus relates to a product having a strong anti-glycation capacity.

In addition, it is noted that a bright skin, like a baby skin, is a skin which reflects the light differently. It is smooth, with an improved micro-relief, strained.

Restoring skin volume, firming the derm, is thus also an approach valid for fighting against the wrinkles and the fine lines, to help the skin to recover the freshness of the complexion and a younger appearance.

If one needs to be convinced, one only needs to note the growing number of dermatologists who use intradermal collagen injections of hyaluronic acid. Today, there is a new technology: the lipofilling, which consists of re-injections of grease taken on the patient to stop the tissue vacuums responsible for wrinkles. All these methods present however certain more or less significant disadvantages. In addition to the price of these interventions, grease, collagen, or the hyaluronic acid injected can always be recognized like belonging to not-oneself and thus to cause inevitably undesirable inflammatory reactions. Moreover, this biological material artificially added, will quickly undergo normal enzymatic degradation, which will induce cycles of injections according to increasingly closer frequencies.

The invention being the subject of this patent application also relates to a product which allows mimic the plastic surgery. The aim is to make a cosmetic “lipofilling” by carrying out a cutaneous filling by increasing the adipocyte mass, which, being newly formed by the organism itself, will gum or reduce the importance of the wrinkles while ensuring a perfect tolerance and painless to the consumer. The principle of this cosmetic lipofilling is the stimulation of the lipids synthesis by cutaneous adipocytes. These effects were studied and shown, in vitro and in vivo, and will be clarified in the examples given in this patent application.

One of the goals of this invention is thus to be able to offer a product which has an effectiveness against cutaneous and capillary ageing in general and in particular against the glycation and the reduction of the subcutaneous lipids, and this without notable side effects.

Many natural products, like green tea, are already used in cosmetics and claim to treat the wrinkles and the fine lines of the skin or to firm cutaneous tissues, to restore the freshness of the complexion. But these compositions treat only incompletely and temporarily those morphological disorders and their use is often non-obvious. Green tea for example tends to blacken the finished products, and its activity is limited: properties claimed for the green tea are anti-radicalizing protection and the antioxidant activity, even thinning when the extract of green tea contains a known amount of caffeine.

However, we discovered, quite surprisingly, that kombucha, in topical application, has interesting cosmetic activities which make it possible to fight against the signs ageing, more specifically fighting against glycation, and the various gaps of skin metabolism, improving the cutaneous microrelief.

Kombucha is a tea which has been subjected to a microbiological transformation. This transformation is a fermentation by a yeast symbiot of the genus Saccharomyces nesting within a matrix of polysaccharides produced by a bacteria, Xylinum. The exact composition of this symbiosis (in particular proportions of each species) varies with the geographical and climatic conditions and depends on the wild local subspecies of yeast and bacteria; nevertheless one can list, inter alia, without this list being restrictive: Saccharomyces ludwigii, species Saccharomyces apicalutus, Bacterium xylinoides, Bacterium gluconicum, Schizosaccharomyces pombe, Acetobacter ketogenum, Torula species, Pichia fermentans and other yeasts. In the literature on kombucha, this yeast symbiot and bacteria are also called “mushroom”, “long-life mushroom” and many synonyms. Fermentation by Kombucha is most unusual in that the alcohol produced from sugar by the yeast is transformed, by the bacterium, into various acids: glucuronic, lactic, usnic and, above all, acetic acid, which confer the acidulated taste to the drink. The final pH is between 2.5 and 4. In addition, the bacterium, Xylinum, known as the “acetic acid bacterium” uses the sugar present in the tea and converts the sucrose into microfibrils of cellulose, thus constituting the supporting membrane in which the yeast nests and grows. The products of yeast metabolism are excreted into the kombucha and consist in numerous vitamins such as vitamins B1, B2, B3 and B12, essential co-factors in bacterial growth.

Kombucha, or the “long-life mushroom tea”, is a popular therapeutic remedy known for a long time by various names [FRANK G, 1999]. It is a soft drink with a yellow-amber color and a soft-acid cider taste. Its effect is known and appreciated since generations by many people, especially in the countries of Eastern Asia. The tea used for the manufacture of kombucha can be of any kind and of any origin, it is in particular about Camellia sinensis, sinensis or assamica varieties. All the varieties of green tea, semi-fermented tea, black tea, smoked black tea, yellow tea, dark tea, white tea, herb tea of plants or of fruits, infusion, can be used as a basis for manufacture of the kombucha. One preferentially uses all the varieties of green tea, semi-fermented tea, black tea, smoked black tea, yellow tea, dark tea, white tea (all Camellia sinsensis) and more particularly the black tea.

Sometimes there can be confusion around the “fermented tea” term. Indeed, the black tea is green tea called “fermented”, but this transformation is not exactly a fermentation, it is an oxidation subjected by the tea when certain substrates are put in contact by a mechanical rupture of the cells while breaking or while cutting the sheets. On the other hand, to obtain green tea, immediately after the gathering, to avoid the oxidation of the tanins and to preserve chlorophyll, the sheets must be put slightly under vapor, then the sheets are rolled and dried.

For the manufacture of the kombucha, black tea is the best culture medium: it leads to the greatest concentration of lactic and gluconic acid, offers the best conditions to the growth of the “mushroom”, and is rich in purins.

The “mushroom”, as well as the receipt of the manufacture of the kombucha are transmitted from generation to generation. The manufacturing process of the kombucha is presented in example to illustrate the present invention. The term “kombucha” in the present invention includes the synonyms such as comboucha, cajnyj kvas (Russian), Combuchagetränk (German), Kargasoktee (German), komboecha-drank (Dutch), Kombuchakwass (German), tea-beer and tea-cider (English), etc . . .

In the same way, the symbiot, the “long-life mushroom” is also called: cajnyj grib (Russian), Japanese or Chinese mushroom, miracle mushroom, comboucha, combucha (Japanese), fungojapon, fungus japonicus (pharmaceutical designation), funko cinese (Italian), ganoderma japonicum, japanischer Teepilz (German), kombucha, ling zhi (Chinese), mandschurischer Schwamm, russische Blume, tea fungus kombucha, Wolgaqualle (German), Zauberpilz, etc . . . .

The kombucha produces, in addition to many substances of antibiotic properties not easily definable, above all, glucuronic acid, vitamins B1, B2, B3, B6 and B12 as well as folic acid, and lactic acid D (+).

There are many therapeutic experiments on the kombucha. In the Asian countries and in Russia, kombucha is used since centuries as natural therapeutic means with great success. In addition to the use as a refreshing drink, one notes a great number of diseases fought successfully by the kombucha which goes from the most futile indisposition to the disease most serious [Hagers Handbuch für die pharmaceutische Praxis, 1973, pp 254-256].

The object of this patent application resides in the discovery, and the demonstration, that the kombucha, suitably prepared and used, proves to be a new solution to fight the signs of ageing exposed previously.

During the development of the project which led to this patent application, we found that the beneficial activities of the kombucha on the applications mentioned above are explained by its composition. Kombucha is indeed rich in vitamins, amino acids, sugars, and in epigallocatechin-gallates and tannins (EGCG).

So topical application of kombucha revivals the activity of the whole cutaneous cellular types (keratinocytes, melanocytes, fibroblasts and adipocytes). It boosts also the immunizing activity by the stimulation of Langherans cells, and the activity of the adipocytes until lipogenesis.

Kombucha can thus be regarded as a restructuring agent against ageing, by its nutritive activity.

Indeed, in a surprising way, we observed and showed that kombucha, by topical application, is effective against the glycation. It is thus able to maintain and restore elasticity and suppleness to old skin in an intrinsic and/or extrinsic way.

We also observed and showed that kombucha, by topical application, stimulates the lipid synthesis by the cutaneous adipocytes. One can thus prevent and treat wrinkles and fine lines of the skin, firm cutaneous tissues of the face, of the hips, thighs, restore the freshness of the complexion by the improvement of the cutaneous microrelief . . .

We also observed and showed that kombucha, by topical application, makes it possible to carry out a cosmetic “lipofilling”.

The kombucha, can also ensure a better cutaneous irrigation, regularize melanocytes activity.

Kombucha, used in cosmetic or dermopharmaceutical compositions in the context of the present invention, can be manufactured starting from any source of tea, herb tea, infusion, by fermentation using any mushroom combination, yeasts and bacteria, in particular those referred to above. However, topical application of kombucha has not yet been described. The present invention thus relates to the cosmetic or dermopharmaceutical compositions containing kombucha.

Compositions according to the invention contain a cosmetically or dermatologically acceptable carrier, i.e. compatible with cutaneous tissues. Thus, the composition can be applied to the whole human body, including mucosae and skin appendages.

Kombucha is used for example in a quantity of 0.001% (w/w) to 100% (w/w) of the total weight of the composition, preferentially between 0.01% (w/w) and 20% (w/w).

Compositions are for example lotions, milks or emollient creams; milks or creams for skin care or hair care; make-up-removing cleansing creams, lotions, or milks; foundation tint bases; sun-screen lotions, milks, or creams; artificial suntan lotions, milks, or creams; shaving creams and foams; aftershave lotions; shampoos, lipsticks, mascaras, or nail varnishes.

These compositions can also be presented in the form of lipsticks intended to apply colour or to protect the lips from cracking, or of make-up products for the eyes or tints and tint bases for the face.

When the compositions according to the invention are presented in the form of water-in-oil or oil-in-water emulsions, the fatty phase consists essentially of a mixture of fatty substances obtained by extraction or synthesis, with at least one oil and possibly another fatty substance. The fatty phase of the emulsions may constitute 5 to 60% of the total weight of the emulsion. The aqueous phase of the said emulsions constitutes preferably 30 to 85% of the total weight of the emulsion. The proportion of the emulsifying agent may be between 1 and 20%, and preferably between 2 and 12% of the total emulsion weight. When the compositions according to the invention are presented in the form of oily, oleo-alcoholic, or aqueous-alcoholic lotions they may constitute, for example, sun-screen lotions containing a filter absorbing UV radiation or softening lotions for skin; the oily lotions may in addition constitute foam oils containing oil-soluble surfactant, bath oils, etc. Among the principal adjuvants commonly used in cosmetic that may be present in compositions according to the invention one may cite organic or hydroglycolic solvents, including MP-diol and polyglycerols, fatty substances obtained by extraction or synthesis, ionic or non-ionic thickeners, softeners, opacifiers, stabilizers, emollients, silicones, α- or β-hydroxy acids, antifoaming agents, moisturizing agents, vitamins, perfumes, preservatives, sequestrating agents, colouring agents, gel-forming and viscosity-increasing polymers, surfactants and emulsifiers, other water- or fat-soluble active principles, plant extracts, tissue extracts, marine extracts, sun filters, and antioxidants.

The more particularly preferred mono- or poly-alcohols are chosen from among ethanol, isopropanol, propylene glycol, glycerol, and sorbitol.

As the fatty substance, among mineral oils one may cite liquid petrolatum; among animal oils whale oil, shark oil, seal oil, menhaden oil, halibut liver oil, cod liver oil, tunny-fish oil, turtle oil, neat's foot oil, horse foot oil, sheep's foot oil, mink oil, otter oil, marmot oil, etc.; and among vegetable oils almond oil, wheat germ oil, jojoba oil, sesame oil, sunflower seed oil, palm oil, walnut oil, shea nut oil, shorea oil, macadamia nut oil, blackcurrant seed oil, and the like.

Among the fatty acid esters one may use esters of C₁₂ to C₂₂ acids, saturated or unsaturated, and lower alcohols such as isopropanol or glycerol or aliphatic C₈ to C₂₂ alcohols, straight-chain or branched, saturated or unsaturated, or C₁₀-C₂₂ alkane 1,2-diols.

As the fatty substance one may also cite vaseline, paraffin, lanolin, hydrogenated lanolin, tallow, acetylated lanolin, and silicone oils.

Among waxes one may cite Sipol wax, lanolin wax, beeswax, Candelilla wax, monocrystalline wax, Carnauba wax, spermaceti, cocoa butter, karité nut butter, silicone waxes, hydrogenated oils solidified at 25° C., sucroglycerides, oleates, myristates, linoleates, and stearates of calcium, magnesium, and aluminium.

Among the aliphatic alcohols one may cite lauryl alcohol, cetyl alcohol, myristyl alcohol, stearyl alcohol, palmityl alcohol, oleyl alcohol, and Guerbet's alcohols such as 2-decyltetradecanol or 2-hexyldecanol. As emulsifying agents among the aliphatic polyoxyethylenated alcohols one may cite lauryl, cetyl, stearyl, and oleyl alcohols containing 2 to 20 moles of ethylene oxide, and among the glycerol alkoyl ethers C₁₂-C₁₈ alcohols containing 2-10 moles of glycerol. It may also be useful to include thickeners such as cellulose derivatives, polyacrylic acid derivatives, guar gum, carouba gum, or xanthan gum.

Compositions according to the invention may include various other and additional ingredients, which may be active, functional, conventionally used in cosmetic, personal care or topical/transdermal pharmaceutical products or otherwise. Of course, a decision to include an additional ingredient and the choice of specific additional ingredients depends on the specific application and product formulation. Also, the line of demarcation between an “active” ingredient and an “inactive ingredient” is artificial and dependent on the specific application and product type. A substance that is an “active” ingredient in one application or product may be a “functional” ingredient in another, and vice versa. A particular ingredient might provide substantivity in one formulation, facilitate transdermal application in another, and merely provide proper viscosity in a third. Which of these is functional and which is active is subject to debate. But, regardless of the outcome, the material in question would qualify as an additional ingredient in accordance with the present invention.

Thus, the compositions of the invention may include one or more additional ingredients, which provide some benefit to the object of the composition. Such additional ingredients may include one or more substances such as, without limitations, cleaning agents, hair conditioning agents, skin conditioning agents, hair styling agents, antidandruff agents, hair growth promoters, perfumes, sunscreen and/or sunblock compounds, pigments, moisturizers, film formers, hair colors, make-up agents, detergents, pharmaceuticals, thickening agents, emulsifiers, humectants, emollients, antiseptic agents, deodorant actives, dermatologically acceptable carriers and surfactants.

The choice of the active or of actives depends on the nature of the cosmetic product or care to formulate. For example, solar filters can be used in anti-solar lotions, shampoos, capillary lotions, and so on. For each type of active, one or more ingredients can be present. The compositions of the present invention may contain a plurality of additional ingredients as well. The CTFA Cosmetic Ingredient Handbook, ninth Edition (2002) describes a wide variety of nonlimiting cosmetic and pharmaceutical ingredients commonly used in the skin care industry, which are suitable for use as additional ingredients in the compositions of the present invention. Non-limiting examples of these additional ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, thickeners, and vitamins and derivatives thereof, actives taken among the group consistent of exfoliants, anti-acne actives, vitamin C and its derivatives, vitamins from B1 to B12 and their derivatives, vitamin E and its derivatives, vitamin H, vitamin K, vitamin A and retinoids, peptides, hydroxy acids, the antioxidants, radical scavengers, chelating agents, anti-inflammatory agents, tanning agents, skin clearing agents, anti-cellulite actives, antimicrobic agents, anti-wrinkle agents, agents stimulating the lipolysis, agents stimulating the lipogenesis, anti-stress agents, inhibitors of the proteolysis, in particular inhibitors of the MMP, enzymes, ceramides and their analogues, anti-irritants and actives softening the skin, anti-pollution actives, healing agents, hydrating agents, emollients, anti-solar protecting agents, solar protecting agents and UV filters, firming agents, liposomes. Further skin care and hair care active ingredients that are particularly useful in combination with the kombucha can be found in SEDERMA commercial literature and on the website www.sederma.fr. (herewith incorporated in its entirety).

In any embodiment of the present invention, however, the additional ingredients useful herein can be categorized by the benefit they provide or by their postulated mode of action. However, it is to be understood that the additional ingredients useful herein can in some instances provide more than one benefit or operate via more than one mode of action. Therefore, classifications herein are made for the sake of convenience and are not intended to limit the additional ingredients to that particular application or applications listed.

Compositions according to the present invention can also contain a sufficient quantity of anti-acne actives. Examples of anti-acne actives include the composition called ac.net® (proposed by SEDERMA, France) and its individual components (nordihydroguaiaretic acid, oleanolic acid), and also resorcinol, sulphur, salicylic acid, benzoyl peroxide, the erythromycin, zinc, etc. Other examples of anti-acne agents are described in details in U.S. Pat. No. 5,607,980, granted to McAtee et al., on Mar. 4, 1997.

Compositions according to the present invention may also contain a sufficient quantity of one or more anti-wrinkle agent. Examples of anti-wrinkle agents being appropriate for the use in the compositions according to the present invention include alpha-hydroxy acids like lactic acid and glycolic acid or beta-hydroxy acids like salicylic acid and its derivatives, vitamins, in particular vitamin B3 and all the retinoids. Isoflavones and phytosterols are also particularly appropriate.

Peptides, and in particular of the di-, tri-, tetra-, and pentapeptides and their derivatives can be included in the compositions according to the present invention in sufficient quantity. Peptides can be natural or synthetic. The dipeptides can be, without being limited to this list, Tyr-Arg, Val-Trp, Asn-Phe, Asp-Phe, beta-Ala-His (Carnosine), N-palmitoyl-beta-Ala-His, Tyr-Arg-hexadecylester, and their derivatives. The tripeptides include Gly-His-Lys, Arg-Lys-Arg, His-Gly-Gly, Lys-Phe-Lys, Lys-Phe-Lys and their analogues of conservative substitution, Gly-His-Lys, Gly-Lys-His, Arg-Lily-Arg-NH₂, and their derivatives. The tetrapeptides include Gly-Gln-Pro-Arg (Rigin), Thr-Lys-Pro-Arg (Tuftsin) Lys-Asn-Pro-Tyr, Lys-Asn-Gly-Tyr, Lys-Asn-(D-Pro)-Tyr, Lys-Asn-Pro-Phe, (D-Lys)-Asn-Pro-Tyr, Lys-Gln-Pro-Tyr, Gly-Asn-Pro-(D-Arg), Gly-Asn-Pro-Tyr, (D-Lys)-Asn-Gly-Tyr, (D-Lys)-Gln-Pro-Tyr and (D-Lys)-Asn-Pro-Phe and their derivatives and analogues by conservative substitution. The pentapeptides and hexapeptides can be, without being limited to this list, Lys-Thr-Thr-Lys-Ser, Tyr-Gly-Gly-Phe-X with X=Met or Leu or mixtures, Val-Gly-Val-Ala-Pro-Gly and their derivatives. These peptides will be used in their free forms or N-acylated. In particular, a preferred dipeptide is N-ac-Tyr-Arg-hexadecylester (CALMOSENSINE® of SEDERMA, France). A preferred tripeptide is N-palmitoyl-Gly-His-Lys (BIOPEPTIDE CL of SEDERMA, France), Peptide CK (Arg-Lys-Arg) and Lipospondine (N-elaidoyl-Lys-Phe-Lys) and its analogues of conservative substitution, Peptide CK+ (N-ac-Arg-Lys-Arg-NH₂). A preferred tetrapeptide is the N-palmitoyl-Gly-Gln-Pro-Arg and a preferred pentapeptide is the N-Pal-Lys-Thr-Thr-Lys-Ser, available under name MATRIXYL® of SEDERMA, France.

The compositions according to the present invention may include antioxidants and/or radicals scavengers to protect the skin from the damage caused by an exposure to the UV. Antioxidants/radical scavengers that may be used can be ascorbic acid (vitamin C) and its salts, ascorbic esters of fatty acids, derivatives of the ascorbic acid (for example magnesium ascorbyl phosphate, sodium ascorbyl phosphate, ascorbyl sorbate), tocopherol (vitamin E), tocopherol sorbate, tocopherol acetate, other esters of tocopherol, benzoic acids hydroxy dutylated and their salts, 6-hydroxy acid (available under name TROLOX®), gallic acid and its alkyl esters, in particular propyl gallate, uric acid and its salts and its alkyl esters, sorbic acid and its salts, lipoic acid, amines (for example N,N-diethylhydroxylamine, amino-guanidine), sulfhydryl compounds (for example glutathion), fumaric dihydroxy acid and its salts, lysine pidolate, arginine pidolate, nordihydroguaiaretic acid, bioflavonoides, curcumin, lysin, methionine, proline, superoxide dismutase, Extremozymes like that proposed under name VENUCEANE®) (commercialized by SEDERMA, France), silymarine, tea extracts, grape extracts, melanin or rosemary extracts.

The flavonoïdes being appropriate for the present invention are the flavanones chosen among un-substituted flavanones, mono-substituted flavanones, and their mixtures; the chalcones selected among un-substituted chalcones, monosubstituted chalcones, disubstituted chalcones, trisubstituted chalcones, and their mixtures; flavones chosen among not-substituted flavones, monosubstituted flavones, disubstituted flavones, and their mixtures; one or more isoflavones; the coumarins chosen among not-substituted coumarins, monosubstituted coumarins, disubstituted coumarins, and their mixtures; selected chromones among the un-substituted chromones, the monosubstituted chromones, the disubstituted chromones, and their mixtures; one or more dicoumarols; one or more chromanones; one or more chromanols; their isomers (for example cis/trans isomers); and their mixtures.

Other examples of flavonoids can be found in PCT request No WO 00/62743 appied by Larry R. Robinson et al. on Apr. 19, 2000, published on Oct. 26, 2000. They can be obtained like extracts of natural sources (plants, algae), like products of hemisynthesis or synthesis. Mixtures of flavonoïd compounds can also be used.

Compositions according to the invention can contain a clearing agent. Skin clearing agents include kojic acid, arbutin, ascorbic acid and its derivatives (in particular magnesium ascorbyl phosphate or sodium ascorbyl phosphate), and extracts (in particular citrus unshiu extract, noraporphine derivatives, busserole extracts and mitracarpus, respectively available like MELASLOW®, LUMISKIN® and ETIOLINE® of SEDERMA, France).

Anti-inflammatory agents can be incorporated in compositions according to the present invention, like natural extracts of plants, fungi, algae. For example, ursolic acid, nordihydroguaiaretic acid, kava-kava extract, bacopa monnieri extract (BACOCALMINE® of SEDERMA, France), candelilla wax, bisabolol, aloe vera, plants sterols, camomile, red clover extract (found asSTEROCARE® of SEDERMA, France), and sea whip extracts, can be used. Other useful anti-inflammatory agents include the compounds of liquorice family (of specie Glycyrrhiza glabra), including glycyrrhetic acid, glycyrrhizic acid, and their derivatives (in particular their salts and esters).

When the compositions according to the invention are in the form of dispersions, these may be dispersions of lecithin in water in the presence of a surfactant or they may be aqueous dispersions of lipid spherules consisting of organized molecular layers enclosing an encapsulated aqueous phase. The lipid compounds may be long-chain alcohols and diols, sterols such as cholesterol, phospholipids, cholesteryl sulfate and phosphate, long-chain amines and their quaternary ammonium derivatives, dihydroxyalkylamines, polyoxyethylenated aliphatic amines, long-chain amino alcohol esters, their salts and quaternary ammonium derivatives, phosphate esters of aliphatic alcohols such as hydrogen dicetyl phosphate or its sodium salt, alkyl sulfates such as sodium cetyl sulfate, fatty acids in the form of salts, or else lipids of the type of those described in French Patents Nos. 2 315 991, 1 477 048, and 2 091 516 or in international patent applications WO 83/01571 and WO 92/08685.

As other lipids one may use, for example, lipids containing a lipophilic long chain of 12 to 30 carbon atoms, saturated or unsaturated, branched or straight-chain, for example an oleyl, lanolyl, tetradecyl, hexadecyl, isostearyl, lauryl, or alkoylphenyl chain. The hydrophilic group in these lipids may be ionic or non-ionic. The non-ionic groups may be groups derived from polyethylene glycol. One can also use advantageously, as lipids forming the lamellar phase, polyglycol ethers such as those described in French Patents Nos. 1 477 048, 2 091 516, 2 465 780, and 2 482 128.

The ionic group may advantageously be a group derived from an amphoteric, anionic, or cationic compound.

Some other lipids described in international patent application WO 83/01571 as suitable for the formation of vesicles are glycolipids such as lactosylceramide, galactocerebroside, gangliosides and trihexosylceramide, as well as phospholipids such as phosphatidylglycerol and phosphatidylinositol.

The active substances may be substances of nutritional or pharmaceutical interest or ones having a cosmetic activity. When they are water-soluble they may be dissolved to produce a homogeneous solution or they are in the aqueous phase encapsulated within the vesicles. The water-soluble substances having a cosmetic and/or pharmaceutical activity may be products intended for skin and hair care or treatment, such as for example moisturizers such as glycerol, sorbitol, pentaerythritol, pyrrolidine acid and its salts; artificial suntan agents such as dihydroxyacetone, erythrulose, glyceraldehyde, γ-dialdehydes such as tartaric aldehyde, these products being possibly associated with colouring agents; water-soluble sun filters; antiperspirants, deodorants, astringents, fresheners, tonics, healing products, keratolytics, depilatories, scents; plant tissue extracts such as polysaccharides; water-soluble colorants; anti-dandruff agents; antiseborrheic agents, oxidants such as bleaching agents, for example hydrogen peroxide; and reducing agents such as thioglycolic acid and its salts.

Mention can also be made of vitamins, hormones, enzymes such as superoxide dismutase, vaccines, antiinflammatories such as hydrocortisone, antibiotics, bactericidal agents, cytotoxic agents, or antitumour agents. When the active substances are oil-soluble they may be incorporated in the walls of the vesicles. They may be chosen from the group formed by oil-soluble sun filters, substances intended for improving of the condition of dry or old skin, tocopherols, vitamins E, F, or A or their esters, retinoic acid, antioxidants, essential fatty acids, glycyrrhetinic acid, keratolytics, and carotenoids.

Kombucha can be used in cosmetic compositions in accordance with the invention either as individual additions or as a premix in a suitable excipient, and be in the form of solution, dispersion, emulsion, paste, or powder. It may be included individually or as a premix in vehicles consisting of cosmetic carriers such as macro-, micro-, or nanocapsules, liposomes or chylomicrons, macro-, micro-, or nanoparticles or macro-, micro-, or nanosponges. It may also be adsorbed on organic polymer powders, talcs, bentonites, or other inorganic supports.

It may be used in any form whatsoever, or in a form bound to or incorporated in or absorbed in or adsorbed on macro-, micro-, and nanoparticles, or macro-, micro-, and nanocapsules, for the treatment of textiles, natural or synthetic fibres, wools, and any materials that may be used for clothing or for day or night underwear intended to come into contact with the skin, such as tights, underclothes, handkerchiefs, or cloths, to exert their cosmetic effect via this skin/textile contact and to permit continuous topical delivery.

The present invention also relates to the use of kombucha and the use of cosmetic and dermopharmaceutical compositions containing the same, alone or in combination, like or for the manufacture of cosmetic or dermopharmaceutical compositions for the care of the skin, mucosae, and skin appendages, in particular to prevent the signs of endogenous and/or exogenous ageing, and in particular restore suppleness and elasticity to the skin, to improve its appearence and the feeling of comfort, to carry out a cosmetic “lipofilling”, to firm the skin of the face, hips, thighs, to invigorate the texture of the skin, to recover matter of the skin, to recover the full forms of a young face, restore the volume of the skin, to restore the freshness of the complexion, to increase radiance.

The present invention also relates to the use of kombucha and the use of cosmetic and dermopharmaceutical compositions containing the same, alone or in combination, like or for the manufacture of cosmetic or dermopharmaceutical compositions to treat in particular the wrinkles and/or the fine lines, the cutaneous and/or under-cutaneous sagging of the features of the face, sagging of the skin of the hips, of the thighs, the deterioration (or the collapse) of cutaneous microrelief, the flask skin, the mat skin.

The present invention also concerns use of kombucha, alone or incorporated in cosmetic or dermopharmaceutical compositions for the preparation of medicinal products intended for the care of the skin, mucosae and skin appendages, and in particular for preventing the signs of endogenous and/or exogenous ageing.

Examples are given below as a non-restrictive illustration of implementation of the present invention.

### EXAMPLE NO 1

### Preparation of Kombucha

In a glass container with a broad opening, let ferment sweetened tea, containing at least 5 g of tea (black tea for example) per liter and at least 70 g of sugar per liter, with the symbiont, for example the “long-life mushroom of kombucha” available by the company R. FRANK (in Birkenfeld in Germany), of which one adds a piece from approximately 25 to 100 g/l to the sweetened tea. Place the fermentation container in a hot place during several (5 to 12) days. After filtration the kombucha is ready, and the “mushroom” is collected for following fermentations. It is possible in the preparation of the kombucha to add in the mixture of sweetened tea and mushroom, 10% of kombucha obtained during a preceding manufacture, in order to initialize more easily the fermentation.

### EXAMPLE NO 2

| Day cream | g/100 g |
|---|---|
| Volpo S20 | 2.4 |
| Volpo S2 | 2.6 |
| Prostearyl 15 | 8.0 |
| Beeswax | 0.5 |
| Stearoxy dimethicone | 3.0 |
| Propylene glycol | 3.0 |
| Carbomere | 0.25 |
| Triethanolamine | 0.25 |
| kombucha | 3.0 |
| Water, preservatives, perfume | qs 100 g |

This emulsion is used to illuminate, and firm the skin of the face.

### EXAMPLE NO 3

| Gel | g/100 g |
|---|---|
| Carbomer | 0.3 |
| Propylene glycol | 2.0 |
| Glycerin | 1.0 |
| White petroleum | 1.5 |
| Cylomethicone | 6.0 |
| Crodacol C90 | 0.5 |
| Lubrajel ® MS | 10.0 |
| Triethanolamine | 0.3 |
| kombucha | 10.0 |
| Water, preservatives, perfumes | qs 100 g |

This gel obtained in a extemporaneous way, can be used in daily application on the skin of the face, to increase radiance in particular.

### EXAMPLE NO 4

| Shampoo | | g/100 g |
|---|---|---|
| A | Potassium sorbate | 0.1 |
| | Water | qs 100 g |
| B | Empicol ESB3/M | 30.0 |
| | INCRONAM 30 | 4.0 |
| | CROTHIX Liquid | 2.0 |
| | Phenova | 0.8 |
| C | Sodium Hydroxide | 0.1 |
| | Water | 1.0 |
| D | kombucha | 0.5 |

### EXAMPLE NO 5

| Hair spray | | g/100 g |
|---|---|---|
| A | Water | qs 100 g |
| | Ethanol | 10.0 |
| | Potassium sorbate | 0.1 |
| B | Procetyl AWS | 0.6 |
| | Nipagine | 0.2 |
| | Butylene Glycol | 3.0 |
| C | kombucha | 3.0 |
| D | perfume | 0.1 |

Method: Weigh A. Weight B and heat at 70° C. until dissolution. Add B to A, agitate. Add C and D at 40° C. Homogenize.

### EXAMPLE NO 6

### Anti-Glycation Effect

Principle: The in vitro model consists in serum albumin incubated with fructose at room temperature for 3 weeks. Glycation is monitored through the formation of fluorescent compounds, generally pentosidines or FFI (furoyl-furanyl-imidazoles.

Protocol: Non-enzymatic glycation between serum albumin and fructose is a slow spontaneous reaction that is accelerated by heat.

In our test, serum albumin (at 2%) and fructose (at 100 mM) were incubated in phosphate buffer medium pH 7.4 at 50° C. for one week. The end products of rearrangement after glycation, here FFI, exhibit natural fluorescence that can be quantified (λ_{excitation}=360 nm and λₑₘᵢₛₛᵢₒₙ=460 nm). The reference (Control) glycation value after one week is determined on the serum albumin and fructose incubation medium. The positive control for glycation inhibition is obtained by incubation in the presence of 0.03% aminoguanidine and the test incubations were conducted in the presence of kombucha at various concentrations.

Results: For each fluorescence value determined, the percentage change on the control is calculated. The results shown in the following table are mean values for n=4 replicates.

TABLE 1
Inhibition of ‘AGE’ production (FF1 compounds)
in the presence of kombucha.

| | % ‘AGE’ decrease |
|---|---|
| aminoguanidine 0.03% | 89 ± 5 |
| KOMBUCHA 1% | 71 ± 7 |
| KOMBUCHA 3% | 79 ± 2 |

The results show excellent inhibition of glycation by aminoguanidine, in line with that reported in the literature (BROWNLEE et al., 1986).

With KOMBUCHA, a very marked antiglycation activity was also observed, without a noteworthy dose effect. The ‘AGE’ formed decreased by 71% and 79% in the presence of KOMBUCHA at concentrations of 1 and 3%, respectively.

### EXAMPLE NO 7

### Stimulation of Cultured Adipocyte Differentiation

Principle: Fibroblasts 3T3 L1, in in vitro culture, differentiate under the action of a cocktail of substances (hormonal messengers) to form pre-adipocytes, then adipocytes loaded with lipids.

Culture is conducted in 3 stages: an initial stage of cell multiplication to confluence, a second stage after addition of the differentiation cocktail, to obtain the initial pre-adipocytes (72 hours), and, lastly, the third stage of active differentiation with stimulation of lipogenesis (72 hours): the storage of lipid droplets is then clearly visible under the microscope

Glycerol-3-phosphate dehydrogenase (G3PDH), an indispensable enzyme in triglyceride synthesis, is very strongly expressed during the active lipid storage phase.

The test product is added at the start of the second stage, at the same time as the differentiation cocktail.

Following the incubation period, the G3PDH activity of the control pre-adipocytes is compared with that of the cells incubated in the presence of the test product.

A product promoting differentiation would increase G3PDH activity.

Protocol: Following the induced differentiation (72 hours), the media were changed. The new media did not contain the differentiation cocktail but contained KOMBUCHA at various concentrations. The pre-adipocytes thus remained in contact with KOMBUCHA for 6 days.

At the end of the incubation period, the cells were separated and lysed and the enzymatic activity of the intracellular contents was determined. G3PDH activity was measured in terms of the elimination of NADH (λ=340 nm).

Results: The results shown in table 2 are the mean values determined for n=4 tests conducted. The enzymatic activity values have been normalized on the number of cells.

TABLE 2
Stimulation of cultured adipocyte differentiation: increase in
G3PDH activity of pre-adipocytes exposed to KOMBUCHA at
various concentrations

| | G3PDH activity/10⁶ cells, % vs. control |
|---|---|
| KOMBUCHA 0.3% | 21 ± 7 |
| KOMBUCHA 1% | 57.5 ± 17.7 |
| KOMBUCHA 3% | 136 ± 265 |

KOMBUCHA increased the G3PDH activity in a dose-dependent manner. The increase was very marked and reached 136% for the 3% KOMBUCHA concentration. The increase in G3PDH activity reflects the stimulant potency of KOMBUCHA on adipocyte differentiation.

Morphology of the pre-adipocytes: A microscopy study of the morphology of the cells at the end of the incubation period under exposure to KOMBUCHA showed a more rich and well differentiated adipocyte population, with lipid inclusions, compared to the control cells.

1-11. (canceled)

12. A composition for topical application comprising: kombucha and a cosmetically acceptable carrier mixed with said kombucha.

13. The composition of claim 12, wherein said kombucha is derived from green tea, semi-fermented tea, black tea, smoked black tea, yellow tea, dark tea, white tea, herb tea of plants or of fruits, or an infusion thereof.

14. The composition of claim 13, wherein said kombucha is derived from green tea, semi-fermented tea, black tea, smoked black tea, yellow tea, dark tea or white tea

15. The composition of claim 14, wherein said kombucha is derived from black tea.

16. The composition of claim 12, wherein said kombucha is present in an amount of 0.001% (w/w) to 100% (w/w) of the total weight of said composition.

17. The composition of claim 16, wherein said kombucha is present in an amount of between 0.01% (w/w) and 20% (w/w).

18. The composition of claim 12, wherein said kombucha when mixed with said cosmetically acceptable carrier is in the form of a solution, dispersion, emulsion, paste or powder.

19. The composition of claim 12, wherein said kombucha may be part of, or contained within, a macrocapsule, microcapsule, nanocapsule, liposome, chylomicron, macroparticle, microparticle or nanoparticle, macrosponge, microsponge or nanosponge, or may be adsorbed on an organic polymer powder, talc, bentonite or inorganic support.

20. The composition of claim 12, wherein said kombucha when mixed with said cosmetically acceptable carrier is in the form of a lotion, milk, emollient, or cream.

21. The composition of claim 12, wherein said mixture of kombucha and a cosmetically acceptable carrier is a formulation for skin care or hair care, a make-up-removing base, a foundation tint base, a sun-screen, an artificial suntan base, a shaving base, an aftershave, shampoo, lipstick, mascara or nail varnish.

22. The composition of claim 12 further comprising an adjuvant, organic or hydroglycolic solvent, fatty substance obtained by extraction or synthesis, ionic or non-ionic thickener, softener, opacifier, stabilizer, emollient, silicone, α- or β-hydroxy acid, antifoaming agent, moisturizing agent, vitamin, perfume, preservative, sequestrating agent, coloring agent, gel-forming or viscosity-increasing polymer, surfactant and emulsifier, other water- or fat-soluble active principle, plant extract, tissue extract, marine extract, sun filter, and antioxidant.

23. The composition of claim 22, wherein said kombucha is present in an amount of 0.001% (w/w) to 100% (w/w) of the total weight of said composition.

24. The composition of claim 23, wherein said kombucha is present in an amount of between 0.01% (w/w) and 20% (w/w).

25. The composition of claim 12 further comprising at least one material useful for the prevention or deceleration of protein glycation.

26. The composition of claim 25, wherein said material useful for the prevention or deceleration of protein glycation is collagen.

27. The composition of claim 12, further comprising an agent useful to stimulate subcutaneous lipid synthesis.

28. An article comprising: the composition of claim 12 bound to, incorporated in, absorbed in or adsorbed on textiles, natural or synthetic fibres, wools, and any materials that may be used for clothing or for day or night underwear intended to come into contact with the skin, such as tights, underclothes, handkerchiefs, or cloths, to exert their cosmetic effect via this skin/textile contact and to permit continuous topical delivery.

29. A method of preventing the signs of endogenous and/or exogenous aging, to restore suppleness and elasticity to the skin, to improve its appearance and the feeling of comfort, to carry out a cosmetic “lipofilling,” to firm the skin of the face, hips, thighs, to invigorate the texture of the skin, to recover matter of the skin, to recover the full forms of a young face, restore the volume of the skin, to restore the freshness of the complexion, to increase radiance to treat in particular the wrinkles and/or the fine lines, the cutaneous and/or under-cutaneous sagging of the features of the face, sagging of the skin of the hips, of the thighs, the deterioration (or the collapse) of cutaneous microrelief, the flask skin, the mat skin comprising the step of applying to skin, mucosae or skin appendage a composition in accordance with claim 12 or 23.
